(19) United States
(12) Patent Application Publication
(10) Pub. No.: US 2005/0165302 A1
(43) Pub. Date: Jul. 28, 2005

(54) Measurements on the visual system of a proband

(76) Inventor: Axel Oeltermann, Tuebingen (DE)

Correspondence Address: BUCHANAN INGERSOLL PC, 1835 MARKET STREET, 14TH FLOOR, PHILADELPHIA, PA, 19103-2985, US

(21) Appl. No.: 10/981,405

(22) Filed: Nov. 4, 2004

(30) Foreign Application Priority Data
Nov. 7, 2003 (DE) DE 103 51 928.9

(51) Int. Cl. A61B 5/05

(52) U.S. Cl. 600/421

(57) ABSTRACT

Described is an eye monitoring device (300) for inductive orientation measuring on an eye (2) of a proband (1), in particular for use in combination with a magnetic resonance tomography unit (200), wherein said eye monitoring device (300) comprises: a transmitting device (320, 310) which is adapted for generating a magnetic field which changes over time, with which magnetic field an induction voltage can be induced in a receiving device (310, 320); a generator device (330) for generating at least one excitation voltage for the transmitting device (320, 310); and a signal processing device (350) for processing induction voltage signals of the receiving device (310, 320), wherein one of the transmitting or receiving devices comprises an eye coil device (310) with an eye coil (311) which can be connected to the eye (2) and which is movable with said eye (2), while the other one of the transmitting or receiving devices comprises a visual-field coil device (320) with at least one visual-field coil (321, 322) which is smaller than the head (3) of a proband (1) and which is equipped during the process of measuring to be arranged so as to be fixed in front of the eye (2), adjacent to a field of view of said eye (2). Also described is an examination device which comprises an eye monitoring device for inductive eye-orientation measuring, as well as a magnetic resonance tomography unit.

The invention relates to an eye monitoring device for inductive orientation measuring on an eye of a proband, in particular for use in combination with a magnetic resonance tomography unit; a visual-field coil device for use in such a eye monitoring device; an examination device which comprises such a eye monitoring device and a magnetic resonance tomography unit; a method for inductive orientation measuring; and a method for magnetic resonance tomography examination of a proband (test subject).

Magnetic resonance tomography as a method for non-invasive imaging is generally known. It is applied in particular in medicine, biology, materials sciences and also in other fields of technology. Generally speaking, a magnetic resonance tomography unit comprises a permanent magnet field device for spin alignment in the sample to be examined, a gradient magnet field device with a gradient coil tube for generating a magnet field gradient which passes through the sample; and a high-frequency coil device for resonant excitation of sample regions in which by the effect of the magnet field gradient a resonance condition is met. For imaging, the gradient of the gradient magnet field in the sample is varied at a specified scanning frequency. Typical operating frequencies of a magnetic resonance tomography unit comprise the scanning frequency, e.g. 10 kHz, and the high frequency for resonance measuring, e.g. 50 MHz to 400 MHz. Further frequencies occur as a result of switching actions in the supply devices of the gradient magnet field device. These switching frequencies have design-specific magnitudes in the kHz range, with harmonic oscillations up to the MHz range.

The quality of magnetic resonance tomography imaging depends in particular on the accuracy with which the various magnetic fields are set and varied in the magnetic resonance tomography unit. It is disadvantageous to the image quality if magnetisable materials are arranged within the gradient coil tube, since such materials can falsify the magnetic fields. For this reason, the arrangement of magnetisable materials in the magnetic resonance tomography unit and in particular in the gradient coil tube is generally avoided in magnetic resonance tomography. Furthermore, up to now the routing of cables in the gradient coil tube has been avoided since voltages can be induced in these cables, which voltages cause interfering magnetic fields. Finally, in particular in the case of magnetic resonance tomography units for carrying out measurements on humans, high frequency shielding is provided which as a rule envelopes the entire space (magnet space) in which the magnetic resonance tomography unit is arranged. To avoid interference, it is thus necessary to keep the magnet space completely free of any additional devices which cause alternating electromagnetic fields.

Investigating the visual system of humans or animals is a promising application of magnetic resonance tomography. For example, in brain-related research, attempts are made, by way of magnetic resonance tomography to localise and quantify brain activities which can be related to visual impressions of the proband. In the investigation of the visual system there is a strong interest, during the magnetic resonance tomography investigation of the proband's brain, in simultaneously presenting an optical stimulus.

However, this method is associated with a problem in that correlations between the presentation of a visual stimulus and the determined sectional images of the brain can reproducibly be evaluated in particular only if the proband's eye has indeed followed the stimulus, and during the measurement was fixed on the stimulus. There is thus the desire to record and quantify the eye movement of a proband during magnetic resonance tomography examinations. However, up to now, no reliable measurement of the direction of view of the proband in the magnetic resonance tomography unit has been available.

Various methods for observing the eye movement of a proband are known. The first method involves inductive orientation measuring, a method which was first proposed by D. A. Robinson in the publication “A method of measuring eye movement using a scleral search coil in a magnetic field” (“IEEE Trans. Biomed. Eng.” vol. BME-10, 1963, pp 137-145). Inductive orientation measuring is based on measuring an induction voltage which is induced in a so-called eye coil by the effect of an external homogeneous alternating electromagnetic field. Up to now, the homogeneous alternating electromagnetic field has been generated with a Helmholtz coil arrangement which comprises several toroidal coils that are arranged all around the head of a proband and which have a diameter of approx. 40 cm. Depending on the orientation of the eye, the eye coil which is integrated in a contact lens worn by the proband, or (in the case of animal experiments) is implanted in the eye, is differently aligned relative to the outer alternating field. With the use of modulation techniques, for directional resolution the amplitude of the voltage induced in the eye coils can be directly correlated with the direction of view of the eye. Measuring of the induction voltage in the eye coil is either line-bound by means of a connection line, or takes place in a non-contacting way by the use of additional induction coils as is described e.g. by L. J. Bour et al. in the publication “The double magnetic induction method for measuring eye movement—results in monkey and men (“IEEE Trans. Biomed. Eng.”, vol. BME-31, 1984, pp 419-427). A contact lens for use in inductive orientation measuring is for example described by H. Collewijn et al. in the publication “Precise recording of human eye movements” (“Vision Research”, vol. 15, 1975, pp 447-450).

Because the Helmholtz coil arrangement takes up a considerable amount of space and, due to the coil size, is sensitive to electromagnetic stray fields, up to now, experts were of the opinion that the applicability of conventional inductive orientation measuring is limited to investigations in shielded, spacious clinical situations.

Video oculography is a further method for measuring eye movements, wherein video images of the eyes are taken by a camera and, for the purpose of recording the direction of view, are evaluated using image processing methods. In the publication by J. N. Vandergeest et al. “Recording eye movements with video-oculography and scleral search coils: A direct comparison of two methods (“Journal of Neuroscience Methods” vol. 114, 2002, pp 185-195) these methods for recording eye movements are compared with each other. There may be a disadvantage of video oculography in that the local resolution and the speed of image evaluation are limited. Video oculography could not be carried out in a magnetic resonance tomography unit because there is not enough room for a video camera with the required spatial and temporal resolution and because such a video camera contains materials and generates operating signals which would severely interfere with the magnetic fields in the magnetic resonance tomography unit.

From practical application, devices are known in which the image of the eye is channelled via a fiberoptic line or via a mirror to a video camera. However, such designs are very expensive. Furthermore, they have the disadvantages of poor angular resolution, very elaborate adjustment and low signal processing speed which is not suitable for recording rapid eye movement.

It is the object of the invention to provide improved eye monitoring devices for inductive orientation measuring on the eye, with which the limitations of conventional eye monitoring devices can be overcome and which offer an expanded scope of application. The eye monitoring devices should in particular require less space and should be less sensitive to interference fields. Furthermore, it is the object of the invention to provide an improved visual-field coil device which makes it possible to achieve a simpler design of an eye monitoring device for inductive orientation measuring. Furthermore, it is the object of the invention to provide an improved examination device for examining the visual system of a proband, which examination device has expanded diagnostic and evaluation options. The object of the invention consists also in the provision of improved methods for inductive orientation measuring and for magnetic resonance tomography examination of probands.

These objects are met with eye monitoring devices, visual-field coil devices, examination devices and methods with the features of claims 1, 11, 23, 29, 39, 45 and 52. Advantageous embodiments and applications of the invention are defined in the dependent claims.

From a device-related point of view, according to a first aspect of the invention, the object is met by the general technical teaching in that, in an eye monitoring device with transmitting or receiving units for inductive eye orientation measuring, one of the transmitting or receiving units is provided with at least one eye coil which can be connected to the eye and is movable with said eye, while the other one of the transmitting or receiving units comprises at least one visual-field coil which is smaller than the head of the proband (test subject) and which is adapted to be arranged so as to be fixed in front of the eye, adjacent to a field of view of the eye during the process of measuring.

In this document, the size of the visual-field coil describes the largest dimension of the coil winding, thus for example the diameter or the length of a toroidal coil or cylindrical coil, or the longer side of a rectangular coil. The size of the visual-field coil can be reduced to such an extent that it is smaller than the head diameter of the proband, thus for example smaller than 25 cm, in particular the same in size as the eye of the proband, thus for example smaller than 5 cm, or smaller still, such as for example 3 cm or below, right into the mm range.

The reduced size of the at least one visual-field coil has the following special advantages. Firstly, the undesirable influence of separate, external magnetic fields can be reduced because they can induce interference voltages only to a reduced extent. Secondly, the eye monitoring device requires less space so that it can be operated also in confined areas such as for example in a magnetic resonance tomography unit. Finally, the danger of undesirably high induction voltages is avoided.

In this document, the term “eye distance of the visual-field coil” denotes the distance between the eye coil and a reference plane which extends so as to be perpendicular to the straight direction of view of the eye and is characteristic of the position of the visual-field coil. The eye distance can also be smaller than the diameter of the head of the proband, in particular smaller than the eye of the proband.

In addition to the reduced space requirement, the reduced eye distance of the visual-field coil, of which there is at least one, provides a particular advantage in that magnetic coupling with the eye coil can be improved. This makes it possible to operate the visual-field coil device at a reduced excitation voltage, so that undesirable interference which may arise in the surroundings as a result of the alternating field of the at least one visual-field coil can be reduced.

In the eye monitoring device according to the invention, the visual-field coil, of which there is at least one, of the visual-field coil device can be arranged so as to be adjacent to a field of view or a range of the field of view of the eye. In this document, the term “field of view” refers to the space angle range which can be viewed by the eye during the inductive orientation measuring process.

With the visual-field coil, of which there is at least one, an inhomogeneous magnetic field can be generated. In a way that is different from the conventional generation of a homogeneous magnetic field, the visual-field coil with an inhomogeneous magnetic field used according to the invention provides an advantage in that the visual-field coil with the reduced size and the reduced distance from the eye with the eye coil can be arranged without this causing any limitation in the processing and evaluation of the induction voltage of the eye coil.

Up to now the belief was commonly held that a homogeneous excitation magnetic field was mandatory, if for no other reason, to be able to carry out the measuring process independently of any head movements of the proband. However, this problem does not arise in the new applications of orientation measuring proposed in this document, for example if the proband is in a prone position or if according to a version of the invention the eye monitoring device or an examination device (see below) comprises a fastening device for holding the head of the proband in place.

Furthermore, advantageously any non-linearity—which may have been introduced as a result of the inhomogeneity of the magnetic field—of the dependence of the induction signal of the receiving unit on the direction of view of the eye can be compensated for after calibration measurements. Calibration measuring involves the recording of correlations between induction signals and specified directions of view, in an advance measuring process, followed by the determination of a two-dimensional compensation model as calibration for the desired orientation measuring.

In the following description, the term “visual-field coil device” in most cases refers to an arrangement of at least one external visual-field coil at a distance from the eye, which coil forms a transmitting coil, while the term “visual eye coil device” refers to a receiving coil in the form of an eye coil on or in the eye. As an alternative, the invention can be implemented in such a way that the functions of the visual-field coil and of the eye coil are the other way round, in other words the eye coil forms a transmitting coil while the visual-field coil forms a receiving coil.

For numerous applications of the invention in which only monitoring of the constancy of the direction of view is desired, the use of a single visual-field coil is sufficient. This can have advantages relating to the compactness of the eye monitoring device. The spacing between the visual-field coil device and the eye coil can in particular in this case be selected to be particularly short (e.g. in the sub-cm range). However, a preferred embodiment of the invention provides for the eye monitoring device to comprise at least two visual-field coils which generate magnetic fields with different spatial orientations (for example horizontally and vertically oriented magnetic fields). In this case, advantages can result in relation to the detection of the direction of view of the eye, in particular of the vertical and horizontal deviation from the view straight ahead.

If the eye monitoring device according to the invention comprises at least two visual-field coils which comprise identical spatial orientations and generate opposing magnetic fields, the following advantages can result in measuring and evaluating the induction voltage of the eye coil. Firstly, with axially-symmetrical arrangement of the coil bodies relative to the straight ahead vision of the eye, the induced voltages in the eye coil are compensated for if said eye coil is aligned so as to be perpendicular to the direction of view. Voltages are induced, but they cancel each other out. If the direction of view changes, the overall voltage changes depending on the angle of view. This dependence corresponds to a sinusoidal curve. The view straight ahead corresponds to the zero crossing of the sinusoidal curve. Thus when the direction of view is changed, strong changes in the induction voltages result and (in the case of small angles of view) an almost linear correlation between the induction voltage and the angle of view results. Secondly, advantageously, opposing winding provides a symmetrical alignment without a preferred field direction relative to the eye coil. As a result of this, the linearity of the inductive orientation measuring procedure applied according to the invention is improved.

Preferably, visual-field coils for generating the above-mentioned opposing magnetic field comprise identical numbers of windings and opposing winding directions so that they can be excited with the same excitation voltages. As an alternative, both coils could be coiled in the same sense of coiling and each coil could have its own generator. This may result in an advantage in that non-linearities or maladjustments can be compensated for by an adjustable different amplitude of the individual excitation voltages.

Particularly preferred is an embodiment of the invention in which the visual-field coil device comprises four visual-field coils with two horizontal coils coiled in opposite directions, and two vertical coils coiled in opposite directions since with an axially-symmetrical arrangement of the visual-field coil device relative to the straight direction of view the above-mentioned advantages of separating the various spatial directions and the linear dependencies of the induction voltage are particularly pronounced. Further advantages in relation to the compactness of the visual-field coil device can result if according to a further modification the visual-field coils are arranged so as to be perpendicular to the straight direction of view in a plane so as to be adjacent to a rectangular window which surrounds the field of view of the eye.

A further preferred embodiment of the invention is characterised by the eye monitoring device being connected with an optical stimulator device which during orientation measuring according to the invention is used for presenting an optical stimulus. Advantageously, the visual-field coil, of which there is at least one, is arranged on a part of the optical stimulator device. The stimulator device, such as for example a housing part or a projector part of said stimulator device, forms a carrier of the visual-field coil device. This variant can have further advantages in relation to a compact design of the eye monitoring device.

Generally, the stimulator device can be formed by an image generator which for example comprises a fixed image representation, a ground glass screen with point projection or image projection, a mirror arrangement or a lightguide arrangement. The part of the image generator which in the operational state points towards the eye can advantageously be used as a coil carrier of the visual-field coil. If the optical stimulator device is formed by a video projection system, its projector can be used as a coil carrier so that advantageously the visual-field coil, of which there is at least one, can be arranged so as to be particularly closely adjacent against the field of view of the eye.

According to a further variant of the invention, the eye coil of the eye monitoring device can be inductively connected to the respective transmitting or receiving unit, for example as described by L. J. Bour et al. (see above). In this case, the eye coil is a shortcut coil with, for example, one winding. Advantageously, in this variant, additional connection lines are avoided.

From a device-related point of view, according to a second, independent, aspect of the invention, the object is met by the general technical teaching of providing an eye monitoring device for inductive eye orientation measuring with a generator device for generating at least one excitation voltage for a transmitting device, wherein by means of the generator device each excitation voltage can be generated such that it does not contain a frequency component which equates to an operating frequency of a magnetic resonance tomography unit. This design according to the invention can generally be realised using various types of transmitting devices (visual-field coils or eye coils), for example Helmholtz coils (provided the application offers sufficient space for their placement), the above-mentioned visual-field coil (of which there is at least one), or a single eye coil. The generator device used according to the invention provides an advantage in that mutual interference between the imaging of the magnetic resonance tomography unit and operation of the visual-field coil of the eye monitoring device is avoided.

According to preferred embodiments, the generator device generates at least one excitation voltage whose frequency component differs from at least one of the following frequencies. If the excitation frequency differs from scanning frequencies of a gradient generator of the magnetic resonance tomography unit, interference of the layer allocation in generating sectional images with the magnetic resonance tomography unit is avoided, and conversely, interference during excitation of the eye coil of the eye monitoring device is avoided. If the excitation frequency differs from the resonance excitation frequencies of the magnetic resonance tomography unit, the setting of the resonance condition and the operation of the eye coil are not subject to interference. Finally, if, during operation of the magnetic resonance tomography unit, switching frequencies occur, for example due to switching processes for power-related optimising the gradient generator, interfering overlay can also be avoided with these switching frequencies or their higher harmonics.

Preferably, the generator device generates excitation frequencies in the frequency interval between the scanning frequency of the gradient generator and the magnetic resonance excitation frequency. In this case, an interference-proof distance from both operating frequencies of the tomography unit can be kept. Particularly preferably, excitation frequencies in the frequency range from 100 kHz to 10 MHz are selected. This frequency range offers wide distance (approximately a factor of 10) of typical scanning frequencies of the gradient generator (e.g. 10 kHz) so that the following advantages result. Firstly, the excitation frequencies can practically not interfere with imaging. Secondly, the band of scanning frequencies can particularly easily be filtered out of the signal of the eye coil. The respective advantages arise due to the wide distances to the magnetic resonance excitation frequency (for example 300 MHz in the case of a 7T tomography unit).

If by way of a transmitting unit the eye monitoring device comprises at least two visual-field coils which generate magnetic fields that are differently oriented in space, the generator device preferably comprises two separate circuit components to generate excitation voltages for the differently oriented visual-field coils. The circuit components comprise, for example, a horizontal branch and a vertical branch for horizontally and vertically aligned coils. Advantageously, the branches can be made from equal circuit components.

According to a preferred implementation of the invention, each of the circuit components in each branch comprises a clock generator, a divider and phase shifter circuit, and a pulse shaper circuit. With these components the excitation frequencies can advantageously be freely set, with high accuracy, in relation to all operational parameters of interest, namely frequency, phase and amplitude.

If each of the circuit components (for example horizontal branches and vertical branches) is adapted to generate reference signals, advantages can result in relation to processing the induction voltages of the eye coil.

According to an important feature of the invention, the frequency difference of the excitation frequencies for the differently oriented visual-field coils is significantly less than the excitation frequencies themselves. Advantageously, this feature makes it possible to provide a common band-pass filter in a signal processing device for evaluating the voltage signal of the eye coil, with which band-pass filter the voltages induced according to the various orientations can be filtered simultaneously. Particularly preferably, the frequency difference is selected to range from 10 kHz to 200 Hz.

As an alternative, the frequency difference between the excitation frequencies of the differently oriented visual-field coils can be selected to be larger (e.g. 500 kHz), which can have advantages in relation to the inductive disturbance of signal components of different orientations, and can thus have advantages for reduced noise.

If in the eye monitoring device the eye coil is used as a transmitting device, the generator device preferably comprises a clock generator, a divider and phase shifter circuit, and a pulse shaper circuit for generating an excitation voltage for the eye coil. Advantageously, the design of the circuit is simplified in this case.

According to further embodiments of the invention the clock generator can comprise a setting unit for changing the clock frequency, and/or a sweep generator. The setting unit can be of advantage for avoiding, if necessary, a specific interference frequency of the gradient generator of the magnetic resonance tomography unit. Advantageously, the sweep generator makes it possible for the clock generator to supply a frequency which rises and falls over time. If this frequency variation is such that the sweep clock (e.g. 500 Hz) is of significantly lower frequency than the clock frequency, advantageously, undesired interference, e.g. as a result of the gradient generator, can be compensated when averaged over time without this necessitating any adjustment of the clock generator. Advantageously, the reference signal in each branch is also automatically swept simultaneously.

According to a further variant of the invention, the output side of the generator device comprises a frequency filter which is tuned such that high-frequency signal components from operating frequencies of the magnetic resonance tomography unit are not transmitted to the generator device. Advantageously, in this way the coupling of interference signals opposite to the direction of signal formation in the generator device can be avoided.

From a device-related point of view, according to a third aspect of the invention, the above-mentioned object is met by the general technical teaching of providing an eye monitoring device for inductive eye orientation measuring with a signal processing device which is adapted to process, from the induction voltage signals, frequency components which correspond to the at least one excitation frequency of the transmitting device, and to block other frequency components which correspond to working frequencies of a magnetic resonance tomography unit or higher harmonics thereof. This design according to the invention, too, generally can be provided with various types of visual-field coils, for example with Helmholtz coils (provided there is sufficient space), with the visual-field coil, of which there is at least one, or with the eye coil.

The signal processing device used according to the invention offers an advantage in that disturbance in the processing of the voltage signals of the receiving device (eye coil or visual-field coil(s)) by operating the magnetic resonance tomography unit is avoided. This advantage is particularly pronounced if the signal processing device is provided in combination with the above-mentioned generator device. However, the use of said signal processing device on its own with a conventional circuit for exciting the receiving device is also possible.

According to preferred embodiments of the invention, the signal processing device suppresses at least one frequency component which corresponds to at least one of the following frequencies or their higher harmonics: scanning frequencies of the gradient generator; magnetic excitation frequencies; and switching frequencies of the magnetic resonance tomography unit. In each case, advantageously, erroneous evaluations of the eye coil signals can be avoided.

If on its input side the signal processing device comprises a frequency filter which is attuned such that high-frequency signal components from operating frequencies of the magnetic resonance tomography unit are not transmitted to the generator device, a coupling of interference signals can advantageously already be avoided at the input to the signal processing device.

If the eye monitoring device as a transmitting device comprises at least two visual-field coils which generate magnetic fields that are differently oriented in space, and comprises the eye coil as a receiving device, the signal processing device preferably comprises a separator stage which is adapted for separating the voltage signal of the eye coil into various direction fractions. If in a concrete application of the invention there is no interest in directional resolution, the circuit can advantageously be simplified in that there is omitted the separator stage. In this case, simple measuring of the amplitude of the voltage signal can determine whether or not the proband's direction of view has changed, wherein, if applicable, there may also be no need to provide a reference signal.

If the eye monitoring device comprises the eye coil as a transmitting device, and at least two visual-field coils as the receiving device, with said visual-field coils generating magnetic fields which are differently oriented in space, the signal processing device preferably comprises a separator stage for separate processing of voltage signals of the visual-field coils. Advantageously, in this case the circuit design can also be simplified.

Generally, the above-mentioned separator stages can be made from filter circuits, which are known per se, for separating frequency components from a mixed signal. According to a preferred variant of the invention, a synchronous rectifier, also known per se, with analog-multipliers is provided, which are adapted for multiplying the voltage signal of the eye coil with the reference signals of the excitation signals at various orientation-related frequencies. When the synchronous rectifier is used, the separator stage is advantageously simplified.

The signal processing and generator devices according to the invention are associated with a general advantage in that they can be constructed from economical standard electronic components which have been selected in the normal way only with a view to their minimum temperature drift and maximum amplification stability.

From a device-related point of view, according to a further independent aspect of the invention, the object is met by the general technical teaching of providing a visual-field coil device for an eye monitoring device for inductive orientation measuring on an eye of a proband, which visual-field coil device comprises a coil carrier on which at least one visual-field coil is arranged on a rim of a view-through aperture in the coil carrier. Advantageously, the view-through aperture forms a window through which the proband can view a scene, for example an optical stimulus.

Particular advantages of the visual-field coil device according to the invention can result if said visual-field coil device comprises the above-mentioned features of the eye monitoring device according to the invention. An advantageous modification is characterised in that the coil carrier of the visual-field coil device can be positioned onto, for example attached to, a housing part or projector part of an optical stimulator device.

According to a particularly preferred embodiment of the visual-field coil device according to the invention, an adaptation circuit comprising at least one series oscillating circuit is arranged on the coil carrier, with at least one visual-field coil being connected to said series oscillating circuit. The adaptation circuit has a particular advantage in that excitation of the visual-field coil takes place exactly at the amplitude specified by the generator device. Furthermore, the visual-field coil is operated in resonance with capacitances in the adaptation circuit so that advantageously the transmission performance of the visual-field coil, of which there is at least one, is increased.

The visual-field coil device according to the invention provides a general advantage, also in its combined effect with the remaining aspects of the present invention, in that there is no particular requirement concerning the precision of the visual-field coils. The visual-field coils are economically available components.

The use of a square coil as a visual-field coil of an eye monitoring device for inductive orientation measuring on an eye represents an independent object of the invention. Advantageously, temporally variable inhomogeneous magnetic fields for the excitation of an eye coil can be generated with the rectangular coil.

From a device-related point of view, according to a fifth aspect of the invention, the above-mentioned object is met by the general technical teaching of providing an examination device for examining a proband, which examination device is formed by combining a magnetic resonance tomography unit for magnetic resonance imaging of a proband, with an eye monitoring device. The examination device according to the invention provides an advantage in that inductive orientation measuring can be carried out simultaneously with magnetic resonance tomography measuring, in particular imaging on the proband.

Up to now, experts were of the opinion that in a magnetic resonance tomography unit, no additional measuring is possible, in particular using alternating electrical and magnetic fields. In particular, mutual influencing of the magnetic fields of the magnetic resonance tomography unit on the one hand, and of the coil arrangement for inductive orientation measuring on the other hand was thought to pose a problem. In contrast to this, with the present invention, accurate and reproducible measuring of body characteristics is proposed for the first time, wherein such measuring relates for example to metabolism characteristics or activities of nerve cells in the examined body part of a proband with the tomography unit directly relating to the current state of the visual system of the proband.

The implementation of the invention is not limited to measuring particular body parts. However, measuring activities relating to a proband's brain are particularly advantageous for investigating the visual system of the proband. In this case, advantageously a visual-field coil device of the eye monitoring device can be arranged jointly with the head of the proband in a gradient coil tube of the magnetic resonance tomography unit in the direction of view in front of the eye. It can be particularly advantageous if at least one visual-field coil is arranged at a distance from the high-frequency coil of the magnetic resonance tomography unit. In this case, mutual magnetic coupling is particularly small, and mutual interference is avoided.

If according to a preferred variant of the invention, electrical connection lines which are fed through magnetic fields that are generated during operation of the magnetic resonance tomography unit are arranged so as to be free of any loops, or if they comprise coaxial cable, further advantages may result as far as a reduction of undesirable induction voltages in the components of the eye monitoring device is concerned. Loop-free alignment of the connection lines means that in the overall routing of the respective connection line through the magnetic fields no large induction loop with an effective induction voltage is formed. To this effect, in each instance two connection lines which lead to a mutual circuit component are arranged so as to be twisted with each other. Such a twisted arrangement is associated with an advantage in that large induction currents are avoided, which induction currents would otherwise themselves cause interfering fields as well as heat build-up.

The inventor has found that the voltage signals of the receiving device (eye coil or visual-field coil(s)) can be transmitted to the signal processing device without intermediate amplification along a relatively long line length. The generator and the signal processing devices can be arranged at a distance from the magnetic resonance tomography unit, which distance is at least 1 m, preferably however 10 to 20 m, and they can in particular be arranged in a separate room. This results in a significant advantage when operating the examination device according to the invention. While magnetic resonance tomography measuring can be carried out completely automatically, inductive orientation measuring requires an operator who, if applicable, issues instructions to the proband, or changes settings on the eye monitoring device. The operator is to be able to work with the best possible protection, at a large distance from the magnetic resonance tomography unit.

Advantageously, the application of the invention is not restricted to a particular type or a particular design of magnetic resonance tomography units. However, it is advantageous from the point of view of adaptation to various probands if a proband carrier is provided which ensures fixed positioning of the proband's head. For examinations involving humans, the proband carrier is preferably a horizontal bed onto which the head can be placed so as to be fixed.

For animal experiments, for example involving monkeys, preferably a proband seat is provided which comprises a fastening device for the animal head. Using a chair or seat for the proband can also provide advantages concerning a physiological seating position of the animal.

There is a further important advantage, in particular for carrying out measuring activities on humans, in that there is no need for an active electronic circuit to be located in the magnetic space. This reduces the danger that interference emanating from the circuit interferes with the HF coil of the tomography unit. Furthermore, magnetic components in the magnet space are avoided; they could be attracted by the tomography unit and could injure the proband.

According to a particularly preferred embodiment of the invention, the examination device comprises a magnetic resonance tomography unit and the eye monitoring device according to the invention with the described advantageous measures for mutual interference suppression of the operation of the tomography unit and the monitoring operation.

From a method-related point of view, according to a further aspect of the invention, the above-mentioned object is met by the general technical teaching of carrying out a method for inductive orientation measuring on an eye of a proband with the eye monitoring device according to the invention such that the visual-field coil device, which is completely positioned beside the head and in front of the eye, is excited by means of the generator device, and an orientation-dependent induction voltage in the eye coil is induced, or in that, conversely, the eye coil is excited by means of the generator device, and an orientation-dependent induction voltage is induced in the visual-field coil device which is completely positioned beside the head and in front of the eye, wherein, for determining orientation information, the induction voltage is evaluated by means of the signal processing device.

According to a first variant of the invention, orientation information can establish whether, and if applicable for how long, the proband's direction of view has remained unchanged. According to a further variant, orientation information can establish in which direction the eye has moved during the process of inductive orientation measuring.

Advantageously, the method according to the invention is compatible with conventional inductive orientation measuring on the eye, so that during orientation measuring, the generation of a visual stimulus in the field of view in front of the eye can also be provided.

According to a particularly preferred embodiment of the invention, in addition to orientation measuring, imaging measuring on the proband is provided, in which image measuring at least one body part, preferably the head of the proband, is arranged in a magnetic resonance tomography unit. According to the invention, orientation measuring preferably takes place at least during measuring in the tomography unit. Depending on the application, orientation measuring before and/or after tomography can be provided.

An independent general aspect of the invention relates to a magnetic resonance tomography measuring method, in particular for imaging examination of a proband in combination with inductive orientation measuring on at least one eye of the proband.

Below, further advantages and details of the invention are provided in the description of preferred embodiments with reference to the enclosed drawings. The following are shown:

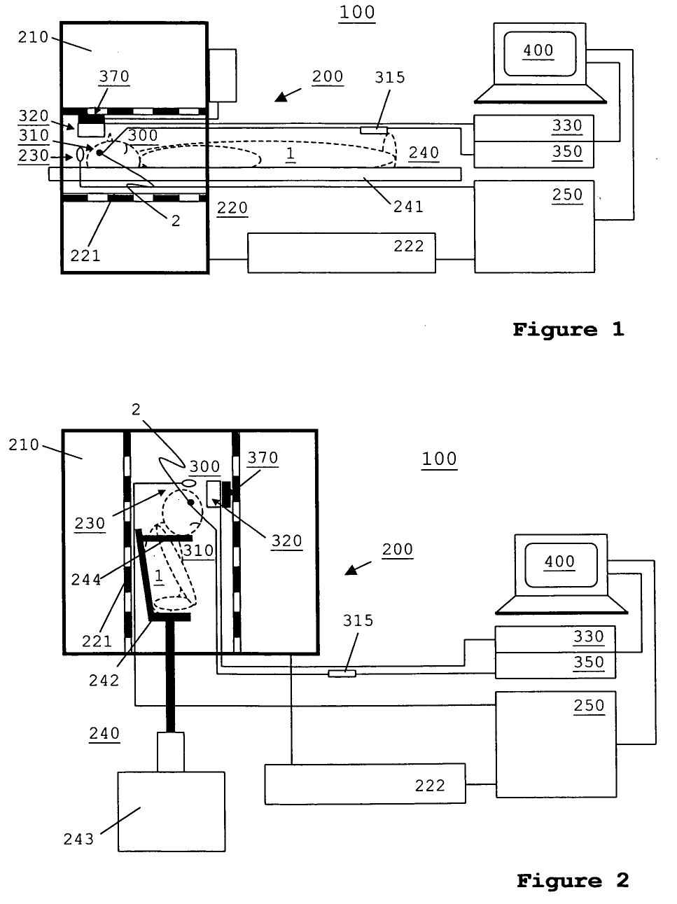

FIGS. 1 and 2: diagrammatic overall views of various embodiments of examination devices according to the invention, for use in magnetic resonance tomography;

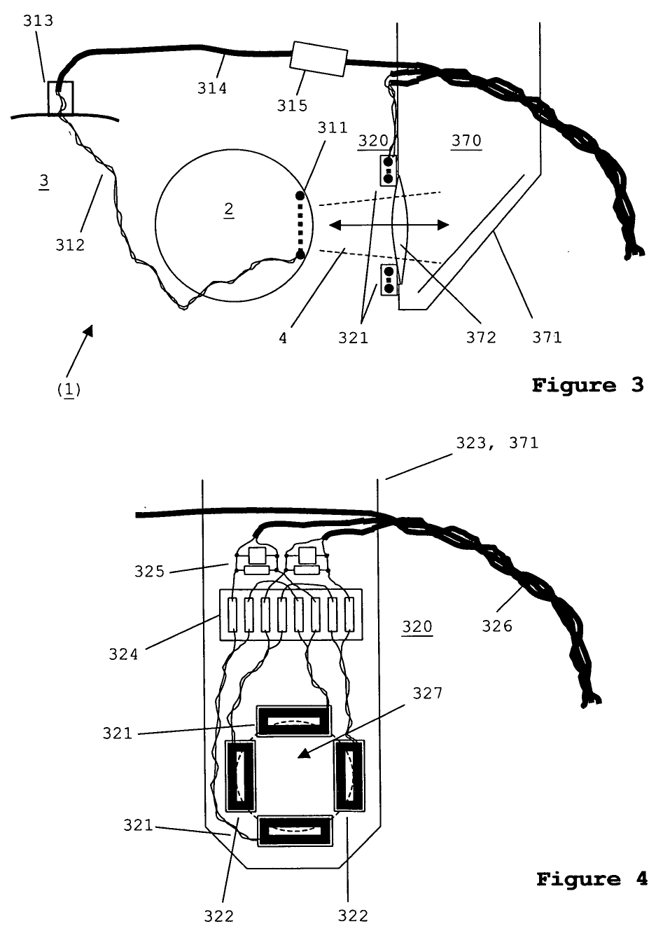

FIG. 3: a diagrammatic view of the mutual alignment of the eye coil device and the visual-field coil device of an eye monitoring device according to the invention;

FIG. 4: a diagrammatic top view of a visual-field coil device used according to the invention;

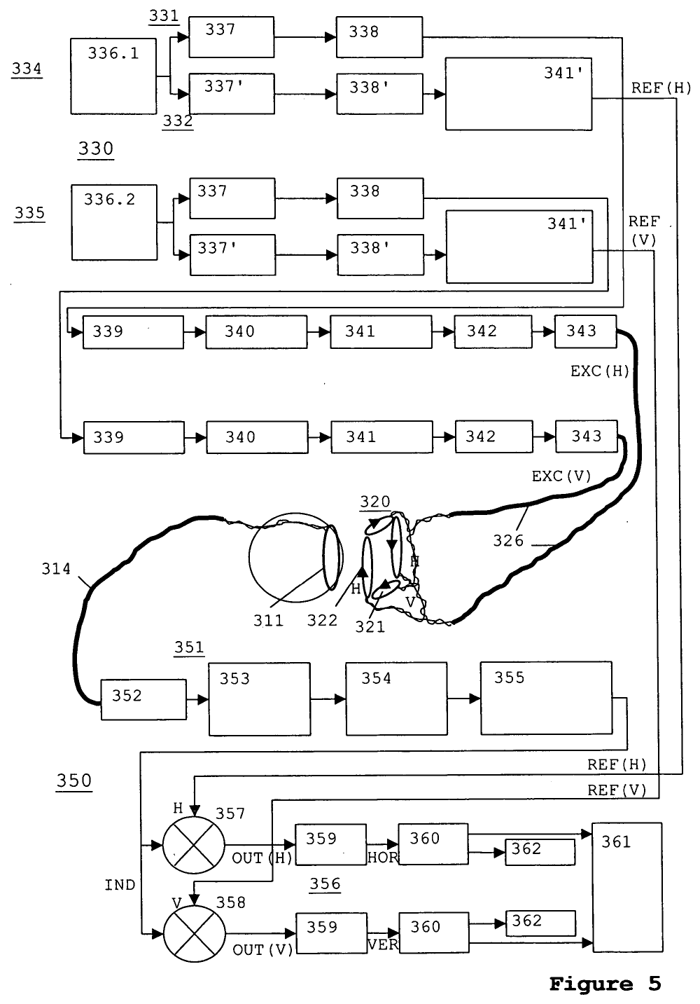

FIG. 5: a block diagram of a first embodiment of an eye monitoring device according to the invention;

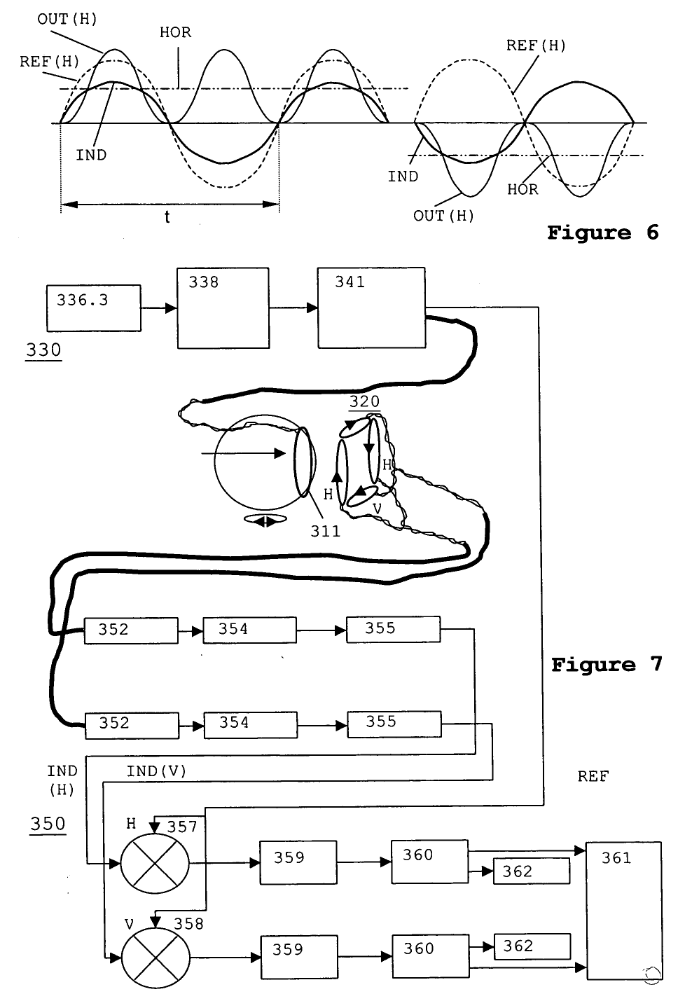

FIG. 6: curves to illustrate an embodiment of the separation, according to the invention, of horizontal signals and vertical signals of the eye-coil device; and

FIG. 7: a block diagram of a further embodiment of an eye monitoring device according to the invention.

FIGS. 1 and 2 show components of an examination device 100 for magnetic resonance tomography with exemplary reference to two embodiments, one of which is preferably adapted for measurements involving human probands (FIG. 1), while the other one is adapted for experiments on animal probands (FIG. 2). It is emphasized that the implementation of the invention is not limited to the diagrammatically illustrated embodiments, but generally is also possible with all other known designs and applications of magnetic resonance tomography units. Although with suitable dimensioning of the magnetic resonance tomography unit the invention can also be implemented with a Helmholtz coil arrangement, which provides the advantage of increased homogeneity of the field in the location of the eye coils, the following visual-field coil device in front of the eye, which device is explained by way of an example, is preferred. The transmitting and receiving functions of the visual-field coil device and the eye coil device can be changed so as to be the other way round, as is shown in FIGS. 3, 4 and 5 on the one hand, and in FIG. 7 on the other hand.

According to FIG. 1, the examination device 100 comprises the magnetic resonance tomography unit 200, the eye monitoring device 300 and a main control device 400, which are described below with further details.

The magnetic resonance tomography unit 200 comprises a permanent magnet field device 210, a gradient magnet field device 220, a high-frequency coil device 230, a proband carrier 240 and a tomography-unit control device 250. By way of an example, these components are realised in a 7T-NMR scanner (manufacturer: Brucker).

The permanent magnet field device 210 is designed in a way which is known per se. It comprises in particular a source for a permanent magnet field, such as e.g. superconductive coils, and if appropriate field-forming devices. The gradient magnet field device 220 comprises a gradient coil tube 221 and a gradient generator (or: gradient amplifier) 222 which is connected to the tomography-unit control device 250 and which serves to excite the gradient coils in the gradient coil tube 221. The gradient coil tube 221 is designed in a way which is known per se. It comprises coil components which have been cast in glass fibre reinforced plastic and which for example comprise an interior diameter of 60 cm.

The high-frequency coil device 230 is also designed in a way which is known per se. It comprises a high-frequency coil 231 which is also connected to the tomography-unit control device 250. The high-frequency coil 231 is a coil with a small diameter (e.g. a few cm), which can be moved within the gradient coil tube 221, or it is a coil of a larger diameter (e.g. a few dm), which is attached to the internal wall of the gradient coil tube 221. In the case of the above-named 7T-NMR scanner, the coil 231 is operated at an excitation frequency of 300 MHz. In a 1.5T-NMR scanner, an excitation frequency of 60 MHz can be provided.

In particular for examining human probands 1, the proband carrier 240 comprises a horizontal bed 241 (FIG. 1). For examining animal probands 1, in particular monkeys, the use of a proband seat 242, for example made from glass fibre reinforced plastic, if appropriate with a fastening device 244 for the head, is preferred (FIG. 2). By means of a servo unit 243, the proband seat 242 can be moved into the tubular structure comprising the permanent magnet field device and the gradient magnet field device (FIG. 2).

The eye monitoring device 300 comprises the eye coil device 310, the visual-field coil device 320, the generator device 330, the signal processing device 350 and the optical stimulator device 370. Reference number 315 relates to a transformer which has been provided for safety reasons so as to prevent any induction voltages in the eye coil device 310. Further details of the above-mentioned components are explained below with reference to FIGS. 3 to 7.

The main control device 400 serves as an interface for operating the examination device 100 by an operator, and is used in particular for inputting and displaying operating parameters and measuring results. The main control device 400 comprises for example one or several computers.

According to FIG. 3, the eye coil device 310 (for example by way of a receiving unit) comprises the eye coil 311, a connection line 312 and a connection plate 313. The example illustrated relates to examinations involving a monkey (macaque), in one eye 2 of which the eye coil 311 is implanted in a way known per se. The connection line 312 subcutaneously leads to the connection plate 313 which has been fixed to the head 3 of the proband monkey 1 and from there by way of a coaxial cable 314 to the signal evaluation device 350 (see FIGS. 1, 2, 5). A transformer 315 has been provided along the length of the coaxial cable 314, preferably on a connection plate outside the permanent magnet of the tomography unit. Advantageously, the transformer 315 provides electrical interruption of the coaxial cable 314 and thus provides safety against unintended induction voltages which could be damaging to the eye of the proband. To avoid interference with the operation of the tomography unit, the transformer 315 is made from non-magnetic materials.

The implanted eye coil 311 for example comprises a diameter of 15 mm and three coil windings. The coil wire comprises stainless steel with a polyethylene insulation (manufacturer: Cooner Wire, USA). The connection line 312 comprises a non-magnetic material, e.g. copper, with an ohmic resistance of 84 Ohm, or comprises the same material as the eye coil.

For measurements on humans the eye coil is integrated into a contact lens in a way which is known per se, wherein said contact lens is placed onto, and fixed to, the eye to be measured.

FIG. 3 illustrates the position of the optical stimulator device 370 in the gradient field of the magnetic resonance tomography unit (not shown) in front of the head 3 of the monkey in the field of view 4 of said monkey. The commercially available video projection system (type: “Silent Vision”) of the manufacturer AVOTEC is used as a stimulator device 370, wherein the projector housing 371 with the exit lens 372 of said video projection system is directed towards the eye 2 in a way which is known per se. By way of the exit lens 372, the visual stimulus, e.g. a dot or circle, is displayed for fixed viewing either as a still dot or circle or as a moving dot or circle. By way of the optical stimulator device 370, an image is presented to the proband 1, which image is viewed during a magnetic resonance tomography examination. The projector housing 371 serves at the same time as a carrier of at least the coils of the visual-field coil device 320 (for example as a transmitting device), with a diagrammatic top view of said visual-field coil device 320 being shown in FIG. 4. The distance between the front of the projector housing 371 (or the carrier 323) and the visual-field coils 321 and the eye coil 311 is e.g. 2 cm.

Advantageously, the magnetic coupling between the visual-field coils 321, 322 and the eye coil is stronger than the mutual coupling of the visual-field coils so that mutual interference is avoided.

According to FIG. 4, the visual-field coil device 320 comprises two pairs of visual-field coils with two horizontal coils 321 and two vertical coils 322 which, surrounding the field of view 4 of the eye 2, are arranged on the coil carrier 323 on the border of a window 327. The visual-field coils, which are associated with a vertical or a horizontal direction respectively, are connected in series. They have the same number of windings but are wound in opposite directions.

The visual-field coils 321, 322 are wound from a non-magnetic wire material which provides good electrical conduction, e.g. Cu wire of 0.2 mm diameter. The winding has a rectangular wave form, e.g. with side lengths of 2 cm *0.5 cm. For example eight to ten windings per coil are provided. For the purpose of optimising the magnetic coupling and the coil size, other dimensions and/or other winding numbers can be provided.

The window 327 of the coil carrier 323 can be a free aperture for a free angle of view onto a visual stimulus, or, as shown in the example of FIG. 3, it can be the exit lens 372 of the stimulator device 370. According to a modification, the visual-field coils 321, 322 can be arranged on the side of the stimulator device 370, which side faces away from the eye 2.

Apart from the visual-field coils, a connection plate 324 and an adaptation circuit 325 are arranged on the carrier 323 (FIG. 4). For each pair of horizontal coils 321 and vertical coils 322, the adaptation circuit 325 comprises a parallel oscillating circuit with a capacitor and the eye coil, wherein said parallel oscillating circuit with the terminal resistance of the coaxial cable 326 is tuned to the excitation frequencies provided in the horizontal branches 334 and vertical branches 335 (see below) of the generator device 330.

The visual-field coil device 320 is connected to the generator device 330 by way of the coaxial cable 326. According to an important feature of the invention, both the coaxial cable 326 and the connection lines between the visual-field coils and the connection plate 324 are arranged so as to be twisted. This is advantageous in that induced voltages which are generated by undesirable magnetic fields in the lines mutually cancel each other out.

Below, details of the generator device 330 are explained with reference to the block diagram in FIG. 5. The generator device 330 comprises two circuit components for providing the excitation voltages of the horizontal coils 321 and vertical coils 322, which are also referred to as horizontal branches 334 and vertical branches 335. In the example shown, these branches only differ in relation to the generated horizontal or vertical excitation frequency, while for the remainder they are of identical design. For this reason only the horizontal branch 334 is described in the following.

In the horizontal branch 334, an external clock generator 336.1 for generating a clock frequency is provided, which clock frequency is output as a TTL signal with a frequency of 16 GHz. The clock generator 336.1 can comprise a setting unit for changing the clock frequency (not shown). Furthermore, the clock generator 336.1 can comprise a sweep generator so that the clock generator 336.1 supplies a frequency which over time alternately rises and falls, e.g. within the range of 16 MHz to 17 MHz.

After the clock generator 336.1, the horizontal branch 334 branches off to an excitation generator 331 and a reference generator 332. For providing the horizontal excitation signal EXC at a desired frequency and phase setting, the clock signal of the generator 336.1 is entered into the divider and phase shifter circuit 337. In the circuit 337 frequency division to 1/16 of the clock frequency takes place as does phase setting corresponding to the step type 360°/16. Phase setting is required for superposition induction signals and reference signals in the synchronous rectifier 357, 358 (see below). Further fine tuning of the phase can take place by way of the optionally provided circuit 340 (see below).

In the pulse shaper circuit 338, the pulse sequence with the desired excitation frequency and phase position is subsequently subjected to an amplitude setting. The pulse shaper circuit 338 comprises CMOS gates with which rectangular pulse shapes of a defined pulse height (e.g. 5 Vₚₚ) are generated. The stability of the pulse heights achieved with the pulse shaper circuit 338 is of particular importance to the accuracy of inductive orientation measuring.

Subsequently, in the pulse power circuit 339, the set pulse height is amplified to a fixed amplitude value. The pulse power circuit 339 is provided as an option. Alternatively, setting the amplitude can take place in the output stage 342. Subsequently, also optionally, phase fine adjustment with the analog phase shifter 340 takes place. The analog phase shifter 340 is for example provided for a fine adjustment by +/−15°. After this, on the low-pass filter 341, higher-frequency signal components are filtered out of the square pulse sequence. As a result of filtering, a sinusoidal excitation voltage at specified values of the frequency (in the present example 1 MHz), amplitude and phase position is generated, wherein blocking of the high frequencies additionally provides the advantage of reducing the influence on magnetic resonance tomography imaging. Undesirable interference to the high-frequency coils 231 by excitation of the visual-field coils 321, 322 is avoided. The low-pass filter 341 is designed such that the first and higher harmonics can be filtered out as much as possible. To this effect a low-pass filter of the 7th order is used. Finally, post amplification takes place at the output stage 342 (power output stage). The amplitude of a sinusoidal excitation voltage is for example 3 Vₛₛ.

Generating the sinusoidal excitation voltage with the illustrated elements of the generator device 330 is not mandatory for implementing the invention. As an alternative, methods that are known per se can be used for generating sinusoidal voltages with the defined wave form parameters, in particular conventional amplitude regulation devices or PLL circuits.

Before the sinusoidal excitation voltage EXC(H) is input into the visual-field coil device 320, further filtering with the frequency filter 343 takes place according to the invention. The high-frequency filter 343 is a passive filter with two L-C members which are tuned in such a way that high-frequency signal components which are induced in the visual-field coil device by the high-frequency coil 231 of the magnetic resonance tomography unit 200 (see FIG. 1) are prevented from passing through to the generator device 330. Otherwise the function of the output stage 342 could be adversely affected. In the example described, the frequency filter 343 blocks the frequency 300 MHz.

From the output of the high-frequency filter 343, a coaxial cable 326 leads to the visual-field coil device 320. The induction and capacitance of the coaxial cables are set such that a frequency-independent transmission characteristic is ensured. Preferably 50 ohm cables are used.

The visual-field coils 321 and 322 and the eye coil 311 interact like a transformer whose output signal (induction voltage of the eye coil 311) depends on the orientation (inclination in the different spatial directions) of the eye coil 311 relative to the visual-field coils 321, 322. The induced voltage is input into the signal processing circuit 350 (see below) via the coaxial cable 314.

The reference generator 332 comprises additional circuits 337′, 338′ and 341′ for generating a horizontal reference signal REF(H) for the signal processing circuit 350. The above-mentioned circuit components comprise the respective characteristics of the phase shifter and divider circuit 337, the pulse shaper circuit 338 and the low-pass filter 341.

In the vertical branch 335, the respective circuit components for generating the vertical excitation voltages EXC(V) and a vertical reference signal REF(V) are provided.

The external clock generator 336.2 for generating a vertical clock frequency is operated relative to the clock generator 336.1 with a frequency spacing at a frequency of 16.16 GHz. As a result of this, the vertical branch 335 supplies a sinusoidal excitation voltage at a frequency of 1.01 MHz.

For the purpose of amplifying and separating the directional components of the induction voltage signals of the eye coil 311, the signal processing device 350 comprises an amplifier stage 351 and a separator stage 356 with the following circuit components.

In the amplifier stage 351, first a high-frequency filter 352 is provided, whose function corresponds to that of the high-frequency filter 343 of the generator device 330. The filtered signal is entered into the preamplifier 353, which is optionally provided to improve the signal-to-noise ratio and if applicable is used for setting the signal amplitude for the following amplifier circuits. The preamplifier 353 has for example an amplification factor of 10. Subsequently, in the band-pass amplifier 354 further amplification in the transmission range of approximately 0.95 MHz to 1.05 MHz takes place. Advantageously, in this region the frequency components of both horizontal components and vertical components can pass through. The amplification factor is for example 30. Subsequently, in the case of an amplifier selector 355, which is also provided as an option, post amplification with a settable amplification factor can be provided.

From the amplifier stage 351, the filtered and amplified signal IND of the eye coil 311, which signal still contains both direction fractions, is entered into the separator stage 356. On the input side, the separator stage 356 comprises a synchronous rectifier with two analog multipliers 357, 358. The function of the analog multipliers 357, 358 is illustrated with the signal gradients shown by way of an example in FIG. 6. For example, for the horizontal signal components the analog multiplier 357 receives as input variables the filtered and amplified signal IND of the eye coil 311, and the (horizontal) reference signal REF(H) of the horizontal branch 334 of the generator device 330. The output signal OUT of the multiplier 357 equates to the product from the signals IND and REF(H). For their signal components with the same phase and frequency the multiplication results in a positive OUT-signal which essentially comprises sin² half-waves. For phase-different and frequency-different signal components, positive and negative components arise in the output signal OUT of the multiplier; however, said positive and negative components cancel each other out on average over time. To this effect, the average-value circuit 359 is provided whose output signal HOR represents the time-averaged output signal OUT of the multiplier 357, wherein said output signal HOR is characteristic for the horizontal deviation of the direction of view of the eye 2 from the direction of view straight ahead. If the direction of view changes, the signal curves shown on the right in FIG. 6 result. While the reference signal REF(H) is preserved, a change in the direction of view from left to right (or, analogously, from top to bottom in the case of the vertical fractions) results in signal reversion of the signal IND of the eye coil. Correspondingly, multiplication results in a reversal of the sign at the output signal OUT of the multiplier 357. Likewise, the sign HOR of the output signal of the average-value circuit 360 changes.

The time segment t shown on the left of FIG. 6 covers for example approximately 1 ps. Accordingly, the threshold frequency of the average-value circuit 359 is set to 50 Hz. If the signal frequencies of the eye monitoring device 300 are changed, the time constant of the average-value circuit 359 has to be changed. To this effect the average-value circuit 359 can comprise a setting unit for setting the time constant of the average-value formation over time.

Finally, the averaged signal HOR is input into an offset selector circuit 360 which is used to enter the signal size in relation to operating parameters of the stimulator device 370. To this effect, the display of the main control device 400 (FIG. 1) by way of points shows not only a visual stimulus, to which the proband fixes his eye, but also the signal which has been determined and evaluated by the receiving unit. Manually or automatically by way of programs which are available per se, the offset selector circuit 360 is set such that both points match at least within the framework of specified tolerances. This setting is made multiple times on various positions of the visual stimulus. Reference number 361 designates an interface at which the output signal is transferred to the main control device 400 (see FIG. 1), wherein said main control device 400 for example comprises an analog-digital converter for further signal processing. Reference number 362 designates an overload detector which is used to display undesirably high signal amplitudes.

According to an important advantage of the invention, the functions of the eye coil 311 and the visual-field coils 321, 322 can be reversed. For example, an excitation voltage can be applied to the eye coil 311, which excitation voltage induces a signal voltage in the visual-field coils 321, 322, wherein said signal voltage depends on the orientation of the eye coil 311. In this case the structure of the generator device 330 and of the signal processing device 350 is simplified, as is diagrammatically illustrated in FIG. 7.

According to FIG. 7, the generator device 330 comprises a single clock generator 336.3 for generating a clock signal at a clock frequency of 1 MHz. The pulse shaper circuit 338 transforms the clock signal into a rectangular wave form with a specified amplitude. Subsequently, analogously to the function described above, the sinusoidal excitation voltage for the eye coil 311 is formed at the low-pass filter 341. The induction voltage signals of the visual-field coil device 320 are supplied to the amplifier stage 351 of the signal evaluation device 350 by way of coaxial cables. The amplifier stage 352 comprises two circuit parts in a manner analogous to the above-mentioned horizontal and vertical branches, in each of which on the input side first a high-frequency filter 352 is provided. This is followed by the provision of the filtered and amplified induction voltage signal IND(V) and IND(H) at the band-pass amplifiers 354 and the amplifier selectors 355.

The separator stage 356 functions analogously to the principles described above, wherein in this case a common reference signal REF of the generator circuit 330 is used for both multipliers 357, 358.

The following steps are provided for the intended use of the examination device 100 according to the invention. After positioning the proband on or in the magnetic resonance tomography unit, the head of the proband is fixed and the optical stimulator device is aligned relative to the eye of the proband. This is followed by a magnetic resonance tomography examination of the respective desired body part as well as by inductive orientation measuring. Both these methods are carried out in relation to the control of the magnetic resonance tomography unit and the generation and processing of visual stimuli according to procedures which are known per se.

The features of the invention which have been disclosed in the above description, in the drawings and claims can be of importance both individually and in any combination for implementing the invention in its various embodiments.

List of Reference Numbers

1 Proband
2 Eye
3 Head
4 Field of view
100 Examination device
200 Magnetic resonance tomography unit
210 Permanent magnet field device
220 Gradient coil device
221 Gradient coil tube
222 Gradient generator
230 High-frequency coil device
231 High-frequency coil
240 Proband carrier
241 Horizontal bed
242 Vertical seat
243 Setting unit
244 Fastening device
250 Tomography-unit control device
300 Eye monitoring device
310 Eye coil device
311 Eye coil
312 Connection line
313 Connection plate
314 Coaxial cable
315 Transformer
320 Visual-field coil device
321 Horizontal coils
322 Vertical coils
323 Coil carrier
324 Connection plate
325 Adaptation circuit
326 Coaxial cable
327 Window
330 Transmission generator
331 Excitation generator
332 Reference generator
334 Horizontal branch
335 Vertical branch
336.1, 336.2, 336.3 Clock generators
337 Divider and phase shifter circuit
338 Pulse shaper circuit
339 Pulse power circuit
340 Phase shifter
341 Low-pass filter
342 Output stage
343 High-frequency filter
350 Filter device
351 Amplifier stage
352 High-frequency filter
353 Preamplifier
354 Band-pass amplifier
355 Amplification selector
356 Separator stage
357 Horizontal multiplier
358 Vertical multiplier
358 Average-value circuit
360 Offset selector circuit
361 Output interface
362 Overload detector
370 Stimulator device
371 Projector housing
372 Exit lens
400 Main control device

1. An eye monitoring device for inductive orientation measuring on an eye of a proband in particular for use in combination with a magnetic resonance tomography unit, comprising:
a transmitting device which is adapted for generating a magnetic field which changes over time, with which magnetic field an induction voltage can be induced in a receiving device;
a generator device for generating at least one excitation voltage for the transmitting device; and
a signal processing device for processing induction voltage signals of the receiving device; wherein
one of the transmitting or receiving devices comprises an eye coil device with an eye coil which can be connected to the eye and which is movable with said eye wherein the other one of the transmitting or receiving devices comprises a visual-field coil device with at least one visual-field coil which is smaller than the head of the proband and which is adapted during the process of measuring to be arranged so as to be fixed in front of the eye, adjacent to a field of view of said eye.

2. The eye monitoring device according to claim 1, in which the visual-field coil device comprises at least two visual-field coils which comprise different spatial orientations.

3. The eye monitoring device according to claim 1, in which the visual-field coil device comprises at least two visual-field coils which comprise identical spatial orientations relative to the field of view and which comprise opposite coiling directions.

4. The eye monitoring device according to claim 3, in which the visual-field coil device comprises four visual-field coils with two horizontal coils coiled in opposite directions, and two vertical coils coiled in opposite directions.

5. The eye monitoring device according to claim 4, in which the horizontal coils and vertical coils are arranged with a rectangular shape in a plane, in which a window is formed through which the field of view of the eye can be directed.

6. The eye monitoring device according to claim 1, in which the at least one visual-field coil can be arranged at a distance from the eye, which distance is less than 10 cm, in particular less than 10 mm.

7. The eye monitoring device according to at least one of the preceding claims claim 1, in which the visual-field coil device is arranged on a coil carrier which forms part of an optical stimulator device.

8. The eye monitoring device according to claim 7, in which the coil carrier forms part of a video projection system of the optical stimulator device.

9. The eye monitoring device according to claim 1, in which the eye coil is inductively connected to the respective transmitting or receiving device.

10. The eye monitoring device according to claim 1, in which the generator device for generating the at least one excitation voltage for the transmitting device is such that said at least one excitation voltage does not contain a frequency component which coincides with working frequencies of a magnetic resonance tomography unit.

11. An eye monitoring device for inductive orientation measuring on an eye of a proband, in particular for use in combination with a magnetic resonance tomography unit, comprising:
a transmitting device which is adapted for generating a magnetic field which changes over time, with which magnetic field an induction voltage can be induced in a receiving device;
a generator device for generating at least one excitation voltage for the transmitting device and
a signal processing device for processing induction voltage signals of the receiving device; wherein
one of the transmitting or receiving devices comprises an eye coil device with an eye coil which can be connected to the eye and which is movable with said eye, wherein the generator device for generating the at least one excitation voltage for the transmitting device is such that said at least one excitation voltage does not contain a frequency component which coincides with working frequencies of a magnetic resonance tomography unit.

12. The eye monitoring device according to claim 11, in which the at least one excitation voltage does not contain a frequency component which belongs to the group of working frequencies which contains scanning frequencies of a gradient generator, resonance excitation frequencies or switching frequencies of the magnetic resonance tomography unit or their higher harmonics.

13. The eye monitoring device according to claim 12, in which the at least one excitation voltage comprises an excitation frequency in the frequency interval between the scanning frequency and the resonance excitation frequency.

14. The eye monitoring device according to claim 13, in which the at least one excitation frequency is selected to be in the frequency range of 100 kHz to 10 MHz.

15. The eye monitoring device according to claim 11, in which the transmitting device comprises horizontal coils and vertical coils of the visual-field coil device, and the generator device comprises a horizontal branch and a vertical branch for generating at least two excitation voltages with horizontal and vertical excitation frequencies for exciting the horizontal coils and vertical coils.

16. The eye monitoring device according to claim 15, in which each of the horizontal branches and vertical branches comprises a clock generator, a divider and phase shifter circuit and a pulse shaper circuit.

17. The eye monitoring device according to claim 15, in which each of the horizontal branches and vertical branches is adapted for generating horizontal reference signals or vertical reference signals.

18. The eye monitoring device according to claim 15, in which the difference between the horizontal and vertical excitation frequencies of the horizontal branches and vertical branches is significantly less than the horizontal and vertical excitation frequencies.

19. The eye monitoring device according to claim 18, in which the frequency difference is selected in a range from 10 kHz to 200 Hz.

20. The eye monitoring device according to claim 11, in which the transmitting device comprises an eye coil of the proband, and the generator device comprises a clock generator a divider and phase shifter circuit, and a pulse shaper circuit for generating an excitation voltage for the eye coil.

21. The eye monitoring device according to claim 11, in which the generator device comprises a high-frequency filter on the output side.

22. The eye monitoring device according to claim 11, in which the signal processing device is adapted for blocking working frequencies of a magnetic resonance tomography unit or the higher harmonics of said working frequencies.

23. An eye monitoring device for inductive orientation measuring on an eye of a proband, n particular for use in combination with a magnetic resonance tomography unit, comprising:
a transmitting device which is adapted for generating a magnetic field which changes over time, with which magnetic field an induction voltage can be induced in a receiving device;
a generator device for generating at least one excitation voltage for the transmitting device; and
a signal processing device for processing induction voltage signals of the receiving device; wherein
one of the transmitting or receiving devices comprises an eye coil device with an eye coil which can be connected to the eye and which is movable with said eye wherein the signal processing device is adapted to block working frequencies of a magnetic resonance tomography unit or the higher harmonics of said working frequencies.

24. The eye monitoring device according to claim 23, in which the signal processing device is adapted to block at least one frequency which belongs to the group of operating frequencies which comprises scanning frequencies of a gradient generator, resonance excitation frequencies of the magnetic resonance tomography unit, or switching frequencies of the magnetic resonance tomography unit or their higher harmonics.

25. The eye monitoring device according to claim 24, in which the signal processing device on an input side comprises at least one high-frequency filter.

26. The eye monitoring device according to claim 23, in which the receiving device comprises an eye coil, and the signal processing device comprises a separator stage which is adapted to separate a voltage signal of the eye coil into various directional components.

27. The eye monitoring device according to claim 23, in which the receiving device comprises horizontal coils and vertical coils of a visual-field coil device, and the signal processing device comprises a separator stage which is adapted for separate processing of voltage signals of the horizontal coils and vertical coils.

28. The eye monitoring device according to claim 26, in which the signal processing device comprises a synchronous rectifier.

29. A visual-field coil device for an eye monitoring device for inductive orientation measuring on an eye of a proband, comprising:
a coil carrier with a window and at least one visual-field coil which is arranged on a border of the window.

30. The visual-field coil device according to claim 29, in which the at least one visual-field coil is smaller than the head of the proband and is adapted during the process of measuring to be arranged so as to be fixed in front of the eye, adjacent to a field of view of the eye.

31. The visual-field coil device according to claim 29, in which an inhomogeneous magnetic field can be generated with the at least one visual-field coil on the eye.

32. The visual-field coil device according to claim 29, in which the visual-field coil device comprises at least two visual-field coils which are arranged with different spatial orientations on the coil carrier.

33. The visual-field coil device according to claim 29, in which the visual-field coil device comprises at least two visual-field coils which are arranged on the coil carrier with identical orientation relative to the field of view and which comprise opposite coiling directions.

34. The visual-field coil device according to claim 33, in which the visual-field coil device comprises four visual-field coils with two horizontal coils coiled in opposite directions, and two vertical coils coiled in opposite directions.

35. The visual-field coil device according to claim 34, in which the horizontal coils and the vertical coils are arranged with a rectangular shape in a plane, in which a window is formed through which the field of view of the eye can be directed.

36. The visual-field coil device according claim 29, in which the coil carrier is a part of an optical stimulator device.

37. The visual-field coil device according to claim 29, in which an adaptation circuit comprising at least one series oscillating circuit is arranged on the coil carrier.

38. The use of a rectangular coil as a visual-field coil of an eye monitoring device for inductive orientation measuring on an eye.

39. An examination device for examining a proband, comprising:
a magnetic resonance tomography unit for magnetic resonance tomography examination of a proband; and
an eye monitoring device which is adapted for inductive orientation measuring on the eye of the proband during the examination, and comprises a transmitting device which is adapted for generating a magnetic field which is changeable over time, with which magnetic field an induction voltage can be induced in a receiving device, a generator device for generating at least one excitation voltage for the transmitting device, and a signal processing device for processing induction voltage signals of the receiving device, wherein one of the transmitting or receiving devices comprises an eye coil device with an eye coil which can be connected to the eye and is movable with said eye.

40. The examination device according to claim 39, in which the transmitting and receiving devices are arranged in a gradient coil tube of the magnetic resonance tomography unit beside the a body part or connected to the body part and at a distance from a high-frequency coil of the magnetic resonance tomography unit.

41. The examination device according to claim 39, in which the electrical connection lines in the magnetic resonance tomography unit are arranged so as to be twisted or comprise coaxial cables.

42. The examination device according to claim 39, in which the generator device and the signal processing device are arranged at a distance of at least 1 m from the magnetic resonance tomography unit.

43. The examination device according to claim 39, in which the magnetic resonance tomography unit comprises a proband carrier which comprises a horizontal bed or a proband seat.

44. The examination device according to claim 39, in which the eye monitoring device comprises the characteristics of claim 1.

45. A method for inductive orientation measuring on a body part of a proband, comprising the steps:
positioning the proband and an eye monitoring device according to claim 1 such that one of the transmitting or receiving devices is arranged beside the eye of the proband while the other of the transmitting or receiving devices is connected to the eye and movable with the eye;
activating the generator device so that a magnetic field which changes over time is generated, and an orientation-dependent induction voltage is induced in the receiving unit; and
processing at least one signal of the receiving unit by means of the signal processing device.

46. The method according to claim 45, in which as a result of processing the signal of the receiving device it is determined whether the eye moved during the measuring process.

47. The method according to claim 46, in which as a result of processing the signal of the receiving device it is determined in which direction the eye moved during the measuring process.

48. The method according to claim 45, in which magnetic resonance tomography imaging on the proband takes place, in which imaging a body part of the proband is arranged in a magnetic resonance tomography unit.

49. The method according to claim 48, in which the proband is positioned such that the head of the proband is arranged in the magnetic resonance tomography unit.

50. The method according to claim 45, in which during orientation measuring with an optical stimulator device a visual stimulus is generated.

51. The method according to claim 50, in which orientation measuring takes place during imaging.

52. A method for imaging on a proband, wherein generation of at least one sectional image of a proband by means of a magnetic resonance tomography unit is provided, wherein on at least one eye of the proband inductive orientation measuring by means of an eye monitoring device is provided, wherein with a generator device of the eye monitoring device at least one excitation voltage for a transmitting device is generated, with which a magnetic field which changes over time is generated, with which in a receiving device an induction voltage is induced which is processed by means of a signal processing device, wherein an eye coil device with an eye coil is used as one of the transmitting or receiving devices, wherein said eye coil is connected to the eye and is movable with said eye.

53. The method according to claim 52, in which for inductive orientation measuring at least one excitation voltage for a transmitting device is generated such that the excitation voltage, of which there is at least one, does not contain a frequency component which coincides with working frequencies of a magnetic resonance tomography unit.

54. The method according to claim 52, in which processing of signals of the inductive orientation measuring process comprises frequency filtering in which the working frequencies of the magnetic resonance tomography unit or their higher harmonics are blocked.

55. The method according to claim 52, in which inductive orientation measuring is carried out using the eye monitoring device according to claim 1.

56. The method according to claim 52, in which inductive orientation measuring is carried out using the visual-field coil device according claim 29.
